(19) United States
(12) Patent Application Publication
(10) Pub. No.: US 2025/0375617 A1
(43) Pub. Date: Dec. 11, 2025

(54) NEUROSTIMULATION LEADS WITH REDUCED CURRENT LEAKAGE

(71) Applicant: AXONICS, INC., Irvine, CA (US)

(72) Inventors: Henry Lee, Irvine, CA (US); Prabodh Mathur, Laguna Niguel, CA (US); Guangqiang Jiang, Irvine, CA (US)

(73) Assignee: AXONICS, INC., Irvine, CA (US)

(21) Appl. No.: 19/311,951

(22) Filed: Aug. 27, 2025

Related U.S. Application Data
(63) Continuation of application No. 17/405,721, filed on Aug. 18, 2021, now Pat. No. 12,420,103.
(60) Provisional application No. 63/068,299, filed on Aug. 20, 2020.

(51) Int. Cl. A61N 1/375; A61N 1/05; A61N 1/36; B29C 45/26; B29K 75/00

(52) U.S. Cl. CPC A61N 1/3752; A61N 1/05; A61N 1/36142; B29C 45/261; B29K 2075/00

(57) ABSTRACT

Embodiments include implantable neurostimulation leads configured to reduce current leakage following implantation in a patient's body, and methods of manufacturing such leads. A lead includes a lead body with proximal apertures and distal apertures; a lumen extending through the lead body; electrodes at a distal portion of the lead body and connector interfaces at a proximal portion of the lead body; conductors extending through the lead body, with their proximal ends exiting via respective proximal apertures to couple with a respective connector interfaces, and their distal ends exiting the interior of the lead body via respective distal apertures to couple with a respective electrodes; and an electrically nonconductive filler element for occupying gaps in the interior of the lead body resulting from the exit of conductors from the lead body.

### CROSS-REFERENCES TO RELATED APPLICATIONS

This application is a continuation application of application Ser. No. 17/405,721, entitled NEUROSTIMULATION LEADS WITH REDUCED CURRENT LEAKAGE filed Aug. 18, 2021, which claims the benefit of U.S. Provisional Application No. 63/068,299, entitled NEUROSTIMULATION LEADS WITH REDUCED CURRENT LEAKAGE, and filed Aug. 20, 2020, the entirety of which is incorporated by reference herein.

### RELATED FIELD

The present invention relates to leads for medical devices, and in particular neurostimulation leads, and methods of manufacturing such leads.

### BACKGROUND

Treatments with neurostimulation systems have become increasingly common in recent years. These neurostimulation systems generally have a neurostimulation component (for example, a pulse generator) and one or more interfacing components. The pulse generator may be an implantable pulse generator (IPG) or an external pulse generator (EPG). The interfacing components may include a neurostimulator programmer, which may be a clinician programmer (CP) or a patient remote for example. The neurostimulator programmer may be able to, for example, adjust stimulation parameters, turn stimulation on or off, receive stimulation history from the pulse generator, and/or transmit programming instructions to the pulse generator.

While neurostimulation systems have been widely implemented in treating a number of conditions, various aspects of neurostimulation systems can be improved. One common issue with implanted neurostimulation systems is current leakage from stimulation electrodes of leads as electricity is delivered through the electrodes. Ideally, the actively stimulating electrodes of a lead would deliver to the targeted tissue exactly the amount of electrical current that is transmitted to them by a pulse generator. However, for various reasons such as those explained herein, conditions are often not ideal in the context of implantable leads, particularly when the leads are implanted for extended periods in an environment including fluid (e.g., body fluid). Implantation in such an environment may result in current leakage when electrodes are actively stimulating. Too much current leakage can be problematic, especially over time. For example, increased current leakage over a period of time may require an inordinate number of recalibrations to effectuate a prescribed stimulation therapy. These recalibrations may require additional energy output by a pulse generator, which may reduce battery life of the pulse generator. In order to minimize these issues, there is an outstanding need for leads that reduce current leakage.

### BRIEF SUMMARY

This disclosure generally relates to neurostimulation treatment systems and associated devices and methods, and in particular to reducing current leakage in neurostimulation leads. The embodiments described herein have particular application to sacral nerve stimulation treatment systems configured to treat bladder and bowel related dysfunctions (e.g., inflammatory bowel diseases). It will be appreciated, however, that the present invention may also be utilized for the treatment of pain, or any other suitable indications, such as movement or affective disorders, as will be appreciated by one of skill in the art.

A common issue with implanted neurostimulation systems is current leakage from stimulation electrodes of leads. This may occur, for example, as electricity is delivered through the electrodes. Neurostimulation leads may include a lead body along which one or more electrodes are secured. The electrodes may be coupled to terminals of a pulse generator (e.g., an IPG, an EPG) via one or more conductors that extend along and within a length of the lead body. In some embodiments, current leakage may in part be due to the migration of body fluid into the lead body over time, creating a conductive internal path between electrodes or conductors of the lead. In some embodiments, the conductors of the lead body, which are disposed within an interior of the lead body, are electrically coupled with electrodes of the lead body, which are disposed along an exterior of the lead body. In order to achieve this coupling, the lead body needs to have one or more exit sites allowing the conductors to couple with the electrodes. These exit sites may provide paths for ingress of body fluids from the surrounding environment of the implant location where the lead is secured. These body fluids include electrolytes, and as such, could serve as a pathway for current to leak by conducting away from the intended target (e.g., nerve tissue external to the lead body) and toward the interior of the lead. This may create an alternative or additional current pathway within the interior of the lead between electrodes. Neurostimulation systems are often implanted for extended periods of time, and this current leakage may increase and compound over time as increasing amounts of body fluid migrate into the lead body.

As a result, periodic recalibrations may be required to effectuate optimal therapy at prescribed stimulation levels. For example, electrical output by an electrode may need to be increased to accommodate for current leakage so as to achieve a prescribed stimulation level. Frequent recalibration can prove to be an inconvenience for patients. The need for frequent recalibration may also result in noncompliance from some patients, thereby reducing the efficacy of neurostimulation for this patient. Additionally, the recalibrations typically increase the required energy output by a pulse generator, which may reduce battery life of the pulse generator. This may be especially problematic in an implantable pulse generator (IPG) with a nonrechargeable battery (e.g., requiring surgery for replacement of the battery), but it is also an issue with an IPG having a rechargeable battery (e.g., requiring more frequent charging of the battery). Similarly, current leakage also affects external pulse generators (EPGs), which would need to have batteries replaced or recharged more frequently than would be optimal.

To address these issues, disclosed herein are implantable neurostimulation leads that, among other things, reduces current leakage by minimizing or preventing the ingress of body fluids into the lead body, and methods of manufacturing such a lead.

In some embodiments, an implantable neurostimulation lead includes a lead body having a proximal portion and a distal portion, wherein the lead body has one or more proximal apertures along the proximal portion and one or more distal apertures along the distal portion; a lumen extending through the lead body; one or more electrodes along an exterior of the lead body at the distal portion of the lead body; one or more connector interfaces along the exterior of the lead body at the proximal portion of the lead body, each connector interface configured to engage with a respective connector of a pulse generator; one or more conductors extending through an interior of the lead body, each of the conductors having a proximal end and a distal end, wherein the proximal end exits the interior of the lead body via a respective proximal aperture to couple with a respective connector interface, and wherein the distal end exits the interior of the lead body via a respective distal aperture to couple with a respective electrode, each conductor coupling a respective electrode with a respective connector interface; and a first filler element configured to occupy at least a portion of one or more gaps in the interior of the lead body resulting from the exit of one or more of the one or more conductors from the lead body, wherein the first filler element comprises an electrically nonconductive material.

In some embodiments, the one or more conductors are coiled around the lumen, and wherein the first filler element is coiled around the lumen. In some embodiments, the one or more conductors are coiled around the lumen at a first pitch, and wherein the first filler element is coiled around the lumen at a second pitch, the second pitch substantially the same as the first pitch. In some embodiments, the one or more conductors are coiled around the lumen at a first pitch, and wherein the first filler element is coiled around the lumen at a second pitch, the second pitch being different from the first pitch.

In some embodiments, the first filler element comprises a multi-filar structure. In some embodiments, the one or more electrodes comprises four electrodes and the one or more connector interfaces comprises four connector interface, and wherein the multi-filar structure comprises three filars. In some embodiments, the one or more electrodes comprises a first electrode and a second electrode; and the one or more conductors comprises a first conductor and a second conductor, wherein a distal end of the first conductor exits the interior of the lead body via a first distal aperture and a distal end of the second conductor exits the interior of the lead body via a second distal aperture, the first distal aperture being distal to the second distal aperture; wherein a first filar of the multi-filar structure is sized to extend between a distal location along the interior of the lead body to the first distal aperture, and wherein a second filar of the multi-filar structure is sized to extend between the distal location to the second distal aperture.

In some embodiments, the one or more connector interfaces comprises a first connector interface and a second connector interface; and the one or more conductors comprises a first conductor and a second conductor, wherein a proximal end of the first conductor exits the interior of the lead body via a first proximal aperture and a proximal end of the second conductor exits the interior of the lead body via a second proximal aperture, the first proximal aperture being distal to the second proximal aperture; wherein a first filar of the multi-filar structure is sized to extend between a proximal location along the interior of the lead body to the first proximal aperture, and wherein a second filar of the multi-filar structure is sized to extend between the proximal location to the second proximal aperture.

In some embodiments, the first filler element is disposed at the distal portion of the lead. In some embodiments, the first filler element is disposed at the distal portion of the lead, and a second filler element is disposed at the proximal portion of the lead, the second filler element comprising an electrically nonconductive material.

In some embodiments, the first filler element comprises a polymer material. In some embodiments, the first filler element comprises a polyurethane material. In some embodiments, the lead body comprises a polymer material. In some embodiments, the lead body comprises a polyurethane material.

In some embodiments, the first filler element comprises a solid extrusion. In some embodiments, the first filler element comprising an adhesive (e.g., polyurethane adhesive) or cured thermoplastic or thermoset polymer material that is configured to be injected into the at least one or more gaps in the interior or lumen of the lead body (e.g., viscous liquid that is heat or ultraviolet (UV) cured).

In some embodiments, the first filler element comprises a filar having a length of IO to 60 mm. In some embodiments, the first filler element comprises a filar having a diameter of 0.03 to 0.3 mm. In some embodiments, the first filler element comprises a ribbon-like filar having a width of 0.01 to 2 mm.

Methods for manufacturing implantable neurostimulation leads are disclosed herein. Such a method includes providing a flexible tubular member from a first electrically nonconductive material, wherein the tubular member comprises a distal portion and a proximal portion, and wherein the tubular member comprises one or more distal apertures at the distal portion of the tubular member and one or more proximal apertures at the proximal portion of the tubular member; disposing one or more conductors around an elongate mandrel in a coiled manner; placing the elongate mandrel within an interior of the tubular member; mounting one or more electrodes along an exterior of the distal portion of the tubular member, the electrodes comprising a first electrically conductive material; mounting one or more connector interfaces along an exterior of the proximal portion of the tubular member, the connector interfaces comprising a second electrically conductive material; for each conductor, causing a distal end of the conductor to exit the distal portion of the tubular member via a respective distal aperture to couple with a respective electrode, and causing a proximal end of the conductor to exit the proximal portion of the tubular member via a respective proximal aperture to couple with a respective connector interface; inserting or injecting a first filler element into the interior of the tubular member to occupy at least a portion of one or more gaps in the interior of the tubular member resulting from the exit of one or more of the one or more conductors from the tubular member, wherein the first filler element comprises a second electrically nonconductive material; and removing the elongate mandrel.

In some embodiments, disposing one or more conductors around the elongate mandrel in a coiled manner comprises coiling the one or more conductors around the elongate mandrel at a first pitch. In some embodiments, inserting or injecting the first filler element into the interior of the tubular member comprises coiling the first filler element around the elongate mandrel at a second pitch, the second pitch substantially the same as the first pitch. In some embodiments, inserting or injecting the first filler element into the interior of the tubular member comprises coiling the first filler element around the elongate mandrel at a second pitch, the second pitch different from the first pitch.

In some embodiments, the one or more conductors are pre-coiled, and wherein disposing one or more conductors around the elongate mandrel in a coiled manner comprises sliding the pre-coiled conductors over the elongate mandrel. In some embodiments, the first filler element is pre-coiled, and wherein inserting or injecting the first filler element into the interior of the tubular member comprises sliding the pre-coiled filler element over the elongate mandrel.

In some embodiments, the first filler may comprise a solid extrusion, wherein inserting or injecting comprises inserting the solid extrusion into the interior of the tubular body and the method further comprises securing at least a portion of the one or more conductors and the solid extrusion within the tubular member. In some embodiments, securing may further include causing at least the first electrically nonconductive material to reflow and set so as to secure at least a portion of the one or more conductors and the first filler element within the tubular member.

In some embodiments, the first filler element may comprise an adhesive or cured thermoplastic or thermoset polymer material, wherein inserting or injecting comprises injecting the polymer material into the interior of the tubular body so as to form a seal to secure at least a portion of the one or more conductors and the first filler element within the tubular member.

In some embodiments, the one or more electrodes comprises a first electrode and a second electrode, and wherein the one or more connector interfaces comprises a first connector interface and a second connector interface. In these embodiments, the method may further include placing a first spacer in between the first electrode and the second electrode; placing a second spacer in between the first connector interface and the second connector interface, wherein the first spacer and the second spacer comprise a third electrically nonconductive material; and causing the third electrically nonconductive material to reflow and set so as to seal the one or more distal apertures and the one or more proximal apertures around the distal and proximal ends of the one or more conductors. In some embodiments, the first electrically nonconductive material and the third electrically nonconductive material are substantially the same. In some embodiments, the first electrically nonconductive material, the second electrically nonconductive material, and the third electrically nonconductive material are substantially the same. In some embodiments, the first electrically nonconductive material, the second electrically nonconductive material, and the third electrically nonconductive material comprise a polyurethane material.

In some embodiments, the first filler element is a multi-filar structure. In these embodiments, inserting or injecting the first filler element into the interior of the tubular member comprises: inserting a first filar into the interior of the tubular member; and inserting a second filar into the interior of the tubular member. Multiple filars may allow for granularity in varying the size of the filler element along the lead body. As many filars may be inserted as necessary to achieve the desired granularity in size variance.

In some embodiments, the first electrically nonconductive material comprises a polymer material. In some embodiments, the first electrically nonconductive material comprises a polyurethane material. In some embodiments, the first electrically nonconductive material and the second electrically nonconductive material are substantially the same. In some embodiments, the first electrically nonconductive material and the second electrically nonconductive material comprise a polyurethane material.

In some embodiments, the method may further include welding the distal end of the conductor to one or more respective electrodes; and welding the proximal end of the conductor to one or more respective connector interfaces. In some embodiments, the method may further include forming the one or more distal apertures and the one or more proximal apertures by creating slits in the tubular member.

In some embodiments, the first filler element is inserted or injected at the distal portion of the tubular member, and wherein the method further comprises inserting or injecting a second filler element at the proximal portion of the tubular member, the second filler element comprising a fourth electrically nonconductive material. In some embodiments, the method may further include removing excess material in the interior of the tubular member, on the exterior of the tubular member, or an exterior of the one or more electrodes by machining, grinding, or polishing subsequently to incorporating the first filler element into the lumen of the tubular member.

Methods for manufacturing an implantable neurostimulation lead may also include incorporating a filler element into an interior of a flexible tubular member to occupy at least a portion of one or more gaps in the interior of the tubular member resulting from an exit of one or more conductors from the tubular member via a respective distal aperture to couple with a respective electrode. The flexible tubular member may comprise a first electrically nonconductive material and the filler element comprises a second electrically nonconductive material. In some embodiments, the first electrically nonconductive material and the second electrically nonconductive material comprise a polyurethane material.

In some embodiments, incorporating may comprise inserting, injecting, molding (e.g., injection molding, blow molding, or compression molding), machining, 2D printing, 3D printing, melt extruding, solid-state forming, casting, vacuum forming, or coating the filler element into the interior of the flexible tubular member.

### BRIEF DESCRIPTION OF THE DRAWINGS

FIG. 1 schematically illustrates a nerve stimulation system, which includes a clinician programmer and a patient remote used in positioning and/or programming of both a trial neurostimulation system and a permanently implanted neurostimulation system.

FIG. 2 illustrates an example of a fully implanted neurostimulation system.

FIG. 3 illustrates an example of a neurostimulation system having an implantable stimulation lead, an implantable pulse generator, and an external charging device.

FIG. 4 illustrates an example lead of a neurostimulation system.

FIG. 5 illustrates a close-up view of a distal portion of the lead.

FIG. 6 illustrates a schematic view of the distal portion of the lead with electrodes removed to show apertures for coupling conductors with the electrodes.

FIG. 7 illustrates an example embodiment of the lead with a filler element at the distal end of the lead.

FIG. 8 illustrates an example embodiment of the lead with a filler element at the proximal end of the lead.

FIG. 9 illustrates an example method of manufacturing a neurostimulation lead.

### DETAILED DESCRIPTION

The present disclosure relates to neurostimulation treatment systems and associated devices, as well as methods of manufacturing such treatment systems. The disclosed systems may in some embodiments relate to sacral nerve stimulation treatment systems configured to treat bladder and bowel dysfunctions, including overactive bladder (“OAB”) as well as fecal dysfunctions, and relieve symptoms associated therewith. It will be appreciated, however, that the disclosed systems may also be utilized for any variety of neuromodulation uses, such as fecal dysfunction, the treatment of pain, or any other suitable indications, such as movement or affective disorders, as will be appreciated by one of skill in the art.

### Neurostimulation Indications

Neurostimulation (or neuromodulation, as may be used interchangeably hereunder) treatment systems, such as any of those described herein, can be used to treat a variety of ailments and associated symptoms, such as acute pain disorders, movement disorders, affective disorders, as well as bladder related dysfunction and fecal dysfunction. Examples of pain disorders that may be treated by neurostimulation include failed back surgery syndrome, reflex sympathetic dystrophy or complex regional pain syndrome, causalgia, arachnoiditis, and peripheral neuropathy. Movement orders include muscle paralysis, tremor, dystonia and Parkinson's disease. Affective disorders include depressions, obsessive-compulsive disorder, cluster headache, Tourette syndrome and certain types of chronic pain. Bladder related dysfunctions include, but are not limited to, OAB, urge incontinence, urgency-frequency, and urinary retention. OAB can include urge incontinence and urgency-frequency alone or in combination. Urge incontinence is the involuntary loss or urine associated with a sudden, strong desire to void (urgency). Urgency-frequency is the frequent, often uncontrollable urges to urinate (urgency) that often result in voiding in very small amounts (frequency). Urinary retention is the inability to empty the bladder. Neurostimulation treatments can be configured to address a particular condition by effecting neurostimulation of targeted nerve tissues relating to the sensory and/or motor control associated with that condition or associated symptom.

### Sacral Neuromodulation (SNM) Overview

SNM is an established therapy that provides a safe, effective, reversible, and long-lasting treatment option for the management of urge incontinence, urgency-frequency, and non-obstructive urinary retention. SNM therapy involves the use of mild electrical pulses to stimulate the sacral nerves located in the lower back. Electrodes are placed next to a sacral nerve, usually at the S3 level, by inserting the electrode leads into the corresponding foramen of the sacrum. The electrodes are inserted subcutaneously and are subsequently attached to an implantable pulse generator (IPG). The safety and effectiveness of SNM for the treatment of OAB, including durability at five years for both urge incontinence and urgency-frequency patients, is supported by multiple studies and is well-documented. SNM has also been approved to treat chronic fecal incontinence in patients who have failed or are not candidates for more conservative treatments.

### Example Systems

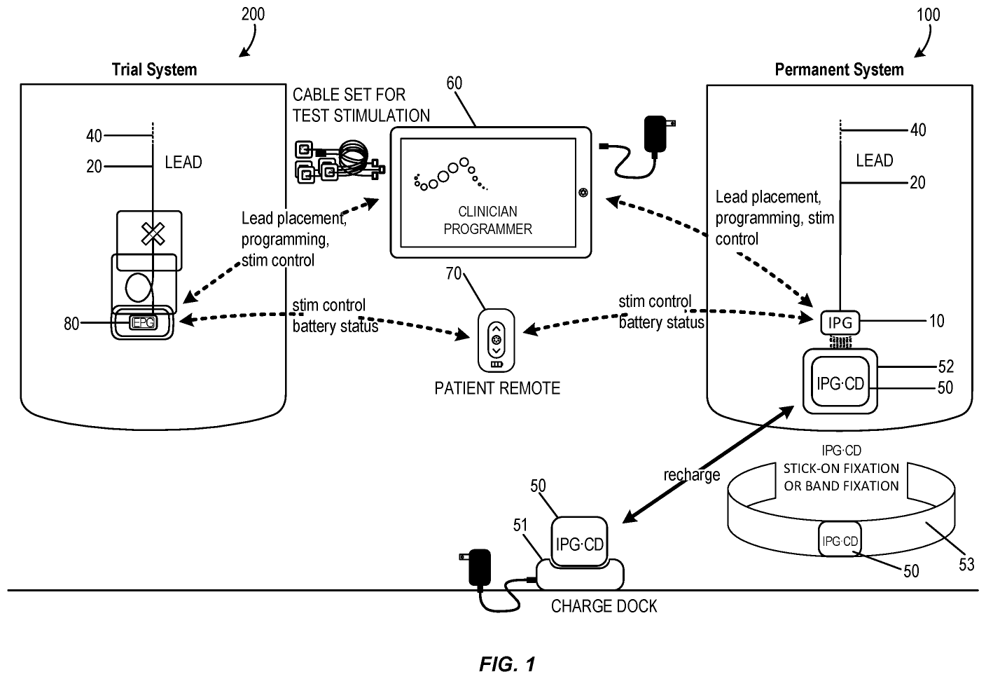

FIG. 1 schematically illustrates example nerve stimulation system setups, which includes a setup for use in a trial neurostimulation system 200 and a setup for use in a permanently implanted neurostimulation system I 00, in accordance with aspects of the invention. The EPG 80 and IPG IO are each compatible with and wirelessly communicate with a clinician programmer (CP) 60 and a patient remote 70, which are used in positioning and/or programming the trial neurostimulation system 200 and/or permanently implanted system I 00 after a successful trial. As discussed above, the system utilizes a cable set and EMG sensor patches in the trial system setup I 00 to facilitate lead placement and neurostimulation programming. CP can include specialized software, specialized hardware, and/or both, to aid in lead placement, programming, re-programming, stimulation control, and/or parameter setting. In addition, each of the IPG and the EPG allows the patient at least some control over stimulation (e.g., initiating a pre-set program, increasing or decreasing stimulation), and/or to monitor battery status with the patient remote. This approach also allows for an almost seamless transition between the trial system and the permanent system.

In one aspect, the CP 60 is used by a physician to adjust the settings of the EPG and/or IPG while the lead is implanted within the patient. The CP can be a tablet computer used by the clinician to program the IPG, or to control the EPG during the trial period. The CP can also include capability to record stimulation-induced electromyograms to facilitate lead placement and programming. The patient remote 70 can allow the patient to turn the stimulation on or off, or to vary stimulation from the IPG while implanted, or from the EPG during the trial phase.

In another aspect, the CP 60 has a control unit which can include a microprocessor and specialized computer-code instructions for implementing methods and systems for use by a physician in deploying the treatment system and setting up treatment parameters. The CP generally includes a graphical user interface, an EMG module, an EMG input that can couple to an EMG output stimulation cable, an EMG stimulation signal generator, and a stimulation power source. The stimulation cable can further be configured to couple to any or all of an access device (e.g., a foramen needle), a treatment lead of the system, or the like. The EMG input may be configured to be coupled with one or more sensory patch electrode(s) for attachment to the skin of the patient adjacent a muscle (e.g., a muscle enervated by a target nerve). Other connectors of the CP may be configured for coupling with an electrical ground or ground patch, an electrical pulse generator (e.g., an EPG or an IPG), or the like. As noted above, the CP can include a module with hardware and computer-code to execute EMG analysis, where the module can be a component of the control unit microprocessor, a pre-processing unit coupled to or in-line with the stimulation and/or sensory cables, or the like.

In other aspects, the CP 60 allows the clinician to read the impedance of each electrode contact whenever the lead is connected to an EPG, an IPG or a CP to ensure reliable connection is made and the lead is intact. This may be used as an initial step in both positioning the lead and in programming the leads to ensure the electrodes are properly functioning. The CP 60 is also able to save and display previous (e.g., up to the last four) programs that were used by a patient to help facilitate re-programming. In some embodiments, the CP 60 further includes a USB port for saving reports to a USB drive and a charging port. The CP is configured to operate in combination with an EPG when placing leads in a patient body as well with the IPG during programming. The CP can be electronically coupled to the EPG during test simulation through a specialized cable set or through wireless communication, thereby allowing the CP to configure, modify, or otherwise program the electrodes on the leads connected to the EPG. The CP may also include physical on/off buttons to turn the CP on and off and/or to turn stimulation on and off.

The electrical pulses generated by the EPG and IPG are delivered to one or more targeted nerves via one or more neurostimulation electrodes at or near a distal end of each of one or more leads. The leads can have a variety of shapes, can be a variety of sizes, and can be made from a variety of materials, which size, shape, and materials can be tailored to the specific treatment application. While in this embodiment, the lead is of a suitable size and length to extend from the IPG and through one of the foramen of the sacrum to a targeted sacral nerve. In various other applications, the leads may be, for example, implanted in a peripheral portion of the patient's body, such as in the arms or legs, and can be configured to deliver electrical pulses to the peripheral nerve such as may be used to relieve chronic pain. It is appreciated that the leads and/or the stimulation programs may vary according to the nerves being targeted.

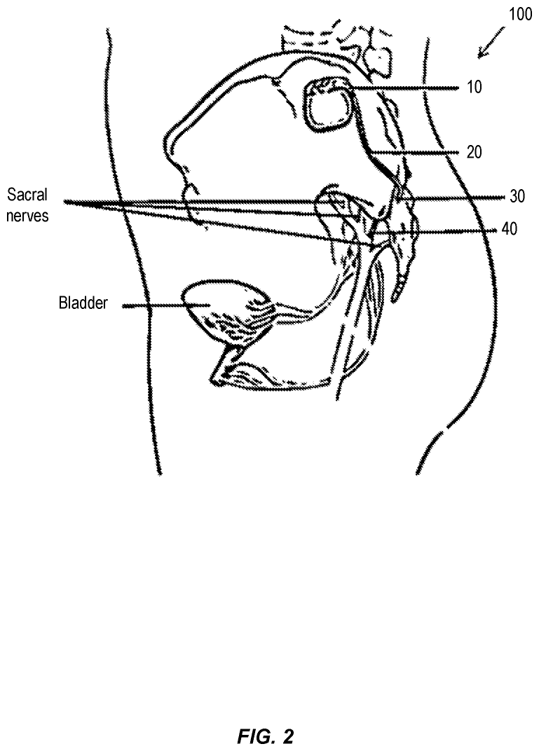

FIG. 2 schematically illustrates an example of a fully implanted neurostimulation system I 00 adapted for sacral nerve stimulation. Neurostimulation system I 00 includes an IPG implanted in a lower back region and connected to a neurostimulation lead extending through the S3 foramen for stimulation of the S3 sacral nerve. The lead is anchored by a tined anchor portion 30 that maintains a position of a set of neurostimulation electrodes 40 along the targeted nerve, which in this example, is the anterior sacral nerve root S3 which enervates the bladder so as to provide therapy for various bladder related dysfunctions. While this embodiment is adapted for sacral nerve stimulation, it is appreciated that similar systems can be used in treating patients with, for example, chronic, severe, refractory neuropathic pain originating from peripheral nerves or various urinary dysfunctions or still further other indications. Implantable neurostimulation systems can be used to cither stimulate a target peripheral nerve or the posterior epidural space of the spine.

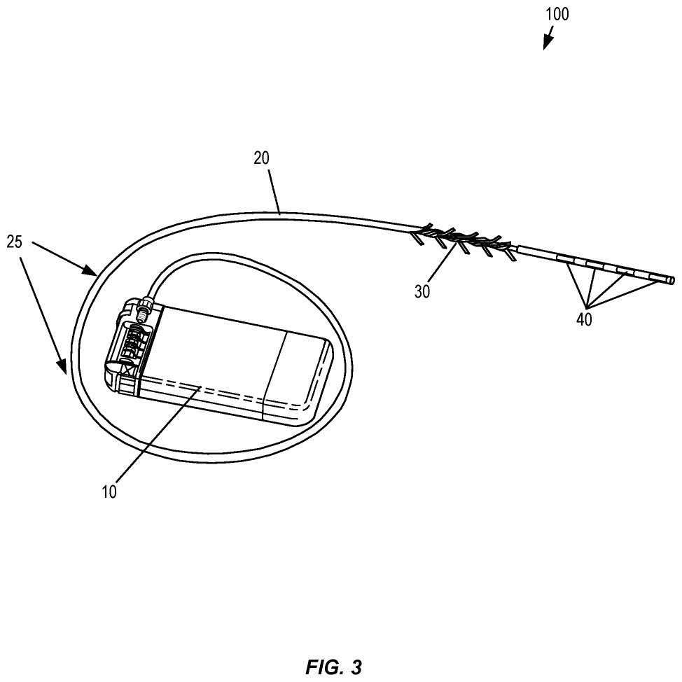

FIG. 3 illustrates an example neurostimulation system 100 that is fully implantable and adapted for sacral nerve stimulation treatment. The implantable system 100 includes an IPG 10 that is coupled to an implantable neurostimulation lead 20 that includes a group of neurostimulation electrodes 40 at a distal end of the lead. The lead includes a lead anchor portion 30 with a series of tines extending radially outward so as to anchor the lead and maintain a position of the neurostimulation lead 20 after implantation. The neurostimulation lead 20 may further include one or more radiopaque markers 25 to assist in locating and positioning the lead using visualization techniques such as fluoroscopy. In some embodiments, the IPG provides monopolar or bipolar electrical pulses that are delivered to the targeted nerves through one or more neurostimulation electrodes. In sacral nerve stimulation, the lead is typically implanted through the S3 foramen as described herein.

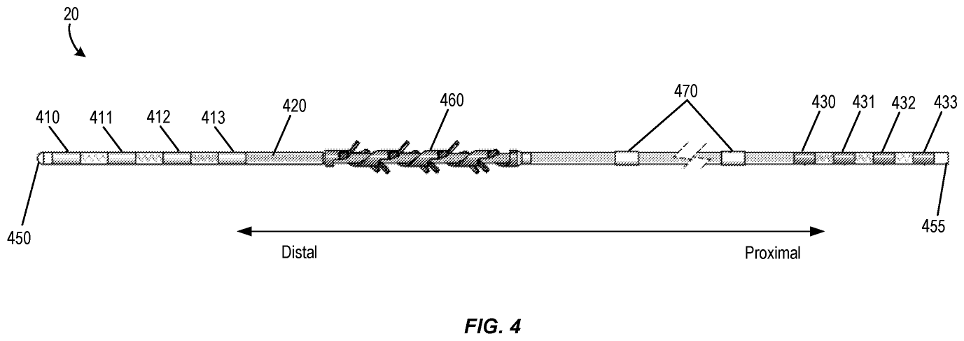

FIG. 4 illustrates an example lead 20 of a neurostimulation system 100. In some embodiments, an implantable neurostimulation lead (e.g., referencing FIG. 4, the lead 20) may include a lead body having a proximal portion and a distal portion. In some embodiments, the lead 20 may be securable to a desired location within the body using a lead anchor 460. The lead anchor 460 may be an integrated anchor that is part of the lead body of the lead 20, or alternatively may be a separate element that is coupleable to the lead 20. In some embodiments, the neurostimulation lead may include one or more electrodes along an exterior of the lead body at the distal portion of the lead body. For example, referencing FIG. 4, the lead 20 includes four electrodes 410, 411, 412, and 413. In this example, the most distal electrode 410 is proximal to the distal end of the lead body (the distal tip 450). The distal tip 450 may be composed of an electrical insulator. Such a configuration may be advantageous in localizing the electrical current delivered by the most distal electrode 410, since the insulated distal tip 450 may reduce or prevent conduction of electrical current along a distal direction beyond the most distal electrode 410. However, the disclosure contemplates that the most distal electrode may alternatively be at the distal end of the lead body. The electrodes may be composed of one or more electrically conductive materials, such as a suitable metal, and may thus act as electrical conductors for stimulating, for example, nerve tissue at a target location.

In some embodiments, the neurostimulation lead 20 may include one or more connector interfaces along the exterior of the lead body at the proximal portion of the lead body. For example, referencing FIG. 4, the lead 20 includes four connector interfaces 430, 431, 432, and 433. In this example, the most proximal connector interface 433 is distal to the proximal end of the lead body (the proximal tip 455). However, the disclosure contemplates that the most proximal electrode may alternatively be at the proximal end of the lead body. Each of these connector interfaces may be configured to engage with a respective connector of a pulse generator. The connectors of the pulse generator may in turn be electrically coupled to the circuitry that is capable of providing an electrical current. Similar to the electrodes, the connector interfaces may be composed of one or more electrically conductive materials, such as a suitable metal, so that they may receive and conduct electrical current from the connectors. Although the disclosure focuses on example leads with four electrodes and four connector interfaces, the disclosure contemplates any number suitable of electrodes and connector interfaces.

In some embodiments, the neurostimulation lead may include one or more conductors extending through an interior of the lead body, each of the conductors having a proximal end and a distal end. The conductors may be made of electrically conductive material, and may couple the electrodes to the connector interfaces. In some embodiments, the distal ends of the conductors may be directly coupled to the electrodes, and the proximal ends of the conductors may be directly coupled to the connector interfaces. For example, referencing FIG. 4, a plurality of conductors 420 may be directly coupled to the electrodes 410, 411, 412, and 413 at the distal ends, and may be directly coupled to the connector interfaces 430, 431, 432, and 433 at their proximal ends. In some embodiments, the conductors may not be coupled directly to the electrodes or connector interfaces, and may instead have one or more intermediary components that electrically couple the conductors to the electrodes or connector interfaces. In some embodiments, each conductor may be electrically insulated from the other conductors (if any), such that connector interfaces are electrically coupled only to respective one or more electrodes. For example, referencing FIG. 4, the plurality of conductors 420 may include four separate conductors that are electrically insulated from each other such that the connector interface 430 is electrically coupled only to the electrode 410, the connector interface 431 is coupled only to the electrode 411, the connector interface 432 is coupled only to the electrode 412, and the connector interface 433 is coupled only to the electrode 413. As another example, a connector interface may be coupled to multiple electrodes (e.g., a single connector interface may be coupled to two electrodes). As another example, an electrode may be coupled to multiple connector interfaces (e.g., a single electrode may be coupled to two connector interfaces).

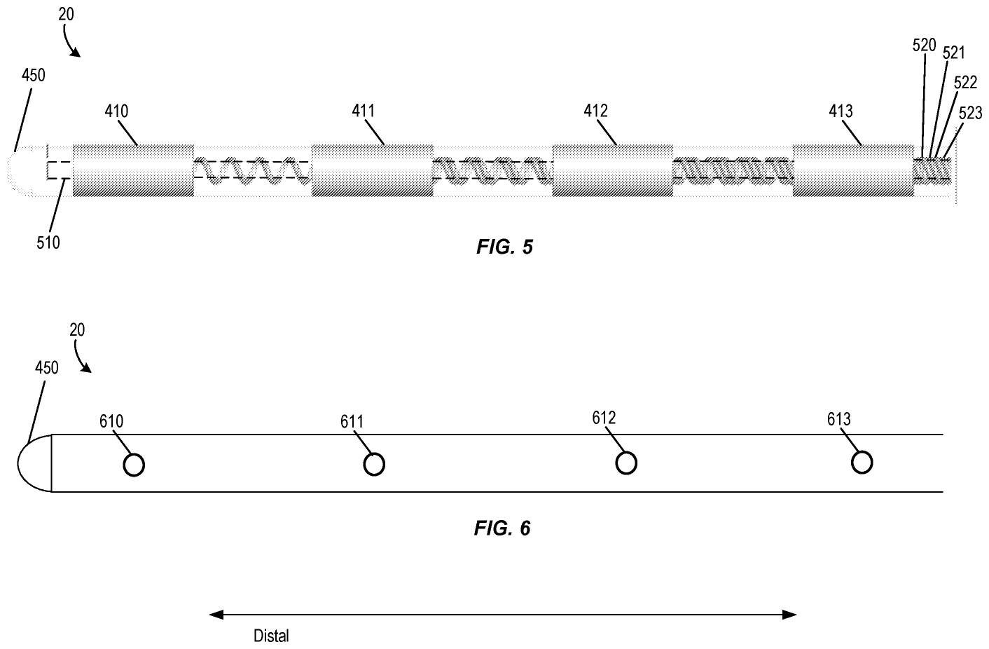

FIG. 5 illustrates a close-up view of a distal portion of the lead 20. In some embodiments, the neurostimulation lead may include a lumen extending through the lead body. For example, referencing FIG. 5, the lead 20 includes a lumen 510 that extends through the lead body. In the illustrated example, the lumen terminates before the distal tip 450 of the lead 20. This lumen may be configured to be able to receive a stylet during the implantation procedure, so that the lead body is afforded sufficient stiffness so as to enable a physician to maneuver the lead body. In some embodiments, the one or more conductors may extend within the lumen. In other embodiments, the one or more conductors may be coiled around the lumen. Referencing FIG. 5, four conductors 520, 521, 522, and 523 are coiled around the lumen 510, each conductor coupled to a respective electrode at the distal end and further coupled to a respective connector interface at the proximal end (not illustrated). In some embodiments, the coiled conductors may be interleaved (e.g., as illustrated in FIG. 5).

In some embodiments, the conductors may be touching each other but still electrically insulated from each other. For example, individual conductors may be surrounded by an electrically insulative coating. Referencing the example illustrated in FIG. 5, the interleaved coiled conductors are catching but nonetheless insulated from each other.

FIG. 6 illustrates a schematic view of the distal portion of the lead 20 with electrodes removed to show apertures for coupling conductors with the electrodes. In some embodiments, the neurostimulation lead may include one or more apertures to enable conductors, which may extend within the interior of the lead body, to couple with the electrodes and/or connector interfaces, which may be disposed along the exterior portions of the lead body. In some embodiments, the lead body may have one or more distal apertures along the distal portion. In some embodiments, each of the distal ends of the one or more conductors may exit the interior of the lead body via a respective distal aperture to couple with a respective electrode. For example, referencing FIG. 6, the apertures 610, 611, 612, and 613 which correspond respectively to electrodes 410, 411, 412, and 413 enable conductors to couple with respective electrodes. The term “apertures” as used herein refers to any suitable opening for allowing a conductor to exit the interior of the lead body. Apertures may be round holes (as illustrated in FIG. 6), rectangular holes, slits, or any other suitable openings. In some embodiments, the distal ends of the conductors may physically exit the interior of the lead body and directly couple with the electrodes. In other embodiments, the distal ends of the conductors may indirectly “exit” the interior of the lead body by coupling with the electrodes via one or more intermediary conductive components that exit the interior of the lead body.

In some embodiments, the lead body may have one or more proximal apertures along the proximal portion. In some embodiments, each of the proximal ends of the one or more conductors may exit the interior of the lead body via a respective proximal aperture to couple with a respective connector interface. The proximal apertures may be similar to the distal apertures described above. The conductors may couple with the connector interfaces similar to how they may couple with the electrodes as described above. That is, in some embodiments, the proximal ends of the conductors may physically exit the interior of the lead body and directly couple with the connector interfaces. In other embodiments, the proximal ends of the conductors may indirectly “exit” the interior of the lead body by coupling with the connector interfaces via one or more conductive intermediary components that exit the interior of the lead body.

In some embodiments, as conductors exit the interior of the lead body from the apertures, they may leave behind gaps in the interior of lead body. These gaps may be fluidically coupled to the exit sites through which the conductors exit the interior of the lead body, and as a result, may be susceptible to an ingress of body fluids from the surrounding environment of the implant location. These body fluids include electrolytes, and as such, could serve as a pathway for current to leak by conducting away from the intended target (e.g., nerve tissue external to the lead body) and toward the interior of the lead. This may create an alternative or additional current pathway within the interior of the lead between electrodes. Neurostimulation systems are often implanted for extended periods of time, and this current leakage may increase and compound over time as increasing amounts of body fluid migrate into the lead body.

As a result, periodic recalibrations may be required to effectuate optimal therapy at prescribed stimulation levels. For example, electrical output by an electrode may need to be increased to accommodate for current leakage within the interior of the lead so as to achieve a prescribed stimulation level. The stimulation level may be described, for example, by the amplitude, frequency, or other parameter of the electrical stimulation that indicates an amount or strength of electrical energy delivered by a pulse generator. Frequent recalibration can prove to be an inconvenience for patients. The need for frequent recalibration may also result in noncompliance from some patients, thereby reducing the efficacy of neurostimulation for this patient. Additionally, the recalibrations typically involve increasing the required energy output by a pulse generator (e.g., increasing the amplitude, frequency, etc.) to compensate for leakage, which may reduce battery life of the pulse generator. This may be especially problematic in an implantable pulse generator (IPG) with a nonrechargeable battery (e.g., requiring surgery for replacement of the battery), but it is also an issue with an IPG having a rechargeable battery (e.g., requiring more frequent charging of the battery). Similarly, current leakage also affects external pulse generators (EPGs), which would need to have batteries replaced or recharged more frequently than would be optimal.

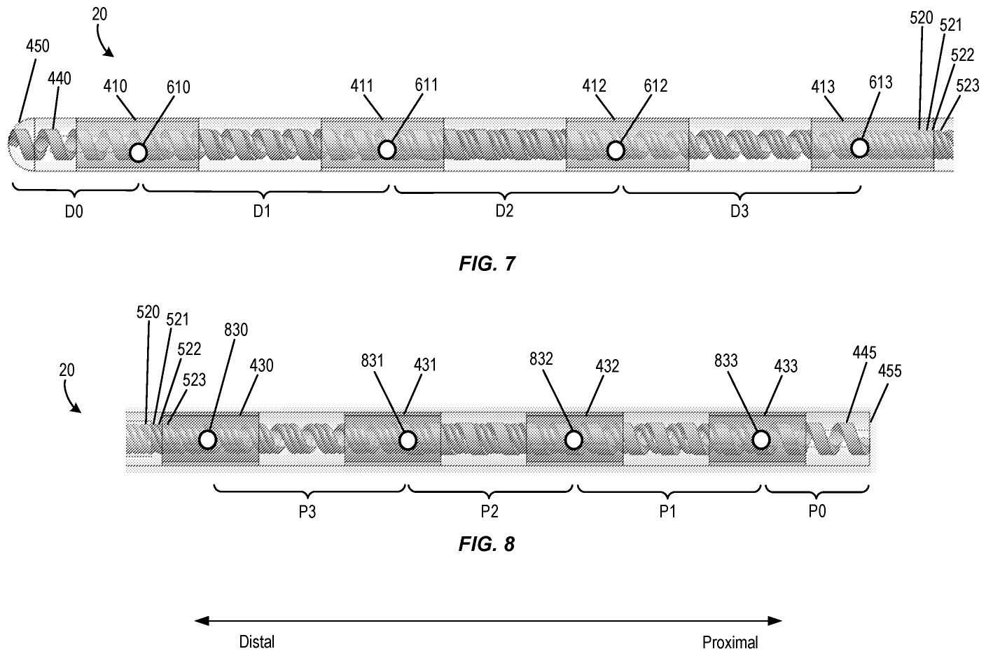

FIG. 7 illustrates an example embodiment of the lead 20 with a filler element 440 at the distal end of the lead 20. In some embodiments, the neurostimulation lead may include a distal filler element configured to occupy at least a portion of one or more gaps in the interior of the lead body. The distal filler element may include an electrically nonconductive material, and may be configured to prevent electrical conduction within the lead body. Referencing the example illustrated in FIG. 7, at segment D3, a gap exists due to the conductor 523 exiting the interior of the lead body via the aperture 613 to couple with the electrode 413; at segment D2, a larger gap exists due to the conductor 522 also exiting the interior of the lead body via the aperture 612 to couple with the electrode 412; at segment DI, an even larger gap exists due to the conductor 521 exiting via the aperture 611 to couple with the electrode 411; and at segment DO, an even larger gap exists due to the conductor 520 exiting via the aperture 610 to couple with the electrode 410. In this illustrated example, the filler element 440 is configured to occupy at least a portion of these gaps. As illustrated in FIG. 7, the filler element 440 extends through segments DO, DI, and D2. In this example, there is no filler element in segment D3, because the gap size in segment D3 may be sufficiently small such that ingress of body fluid would be minimal or negligible. However, in another example embodiment, the segment D3 may also include a filler element. By occupying the gaps, the electrically nonconductive filler element may prevent or reduce the ingress of body fluid into the lead body, and may thus prevent or reduce current leakage. The filler element may be disposed within the interior of the lead body in any suitable configuration, and may be disposed in configurations that optimize the amount of space occupying the gaps. For example, referencing FIG. 7, the filler element 440 may be coiled around the lumen so as to best conform to the gaps left by the coiled conductors as they exit the lead body in the illustrated example. In this example, the one or more conductors may be coiled around the lumen at a first pitch, and the first filler element is coiled around the lumen at a second pitch, the second pitch being substantially the same as the first pitch. Alternatively, the second pitch may be different from the first pitch. In another example lead, where conductors are disposed within the lead body in a substantially straight and uncoiled configuration, the filler element may be substantially straight and uncoiled to best conform to gaps left by the conductors.

In some embodiments, the filler element may be further optimized to account for differences in gap size along the length of the lead body. Referencing the example in FIG. 7, as discussed above, the gap sizes of the different segments of the lead 20 vary, with segment D3 having the smallest gap size (e.g., since only one conductor has terminated or exited the interior of the lead body at this segment) and segment DO having the largest gap size (e.g., since all conductors have terminated or exited the interior of the lead body at the segment). The filler element 440 may be sized to account for this change in gap size. For example, portions of the filler element 440 may be sized to optimally occupy the gaps corresponding to the segments in which they are to be disposed. In this example, the portion of the filler element 440 configured to be disposed in segment DO may be sized to optimally occupy the gap in segment DO, and the portion of the filler element 440 configured to be disposed in segment D2 may be sized to optimally occupy the gap in segment D2. In this example, the portion of the filler element 440 corresponding to segment D2 may have a larger volume than the portion of the filler element 440 corresponding segment DI. As another example, the filler element 440 may include a multi-filar structure with multiple filars to account for varying gap sizes. In this example, referencing FIG. 7 again, the filler element 440 may be a structure including four separate filars to account for varying gap sizes in the four different segments. In this example, a portion of the filler element 440 corresponding to segment DO may include four filars (e.g., to account for four missing conductors), a portion of the filler element 440 corresponding to segment DI may include three filars (e.g., to account for three missing conductors), a portion of the filler element 440 corresponding to segment D2 may include two filars (e.g., to account for two missing conductors), and a portion of the filler element 440 corresponding to segment D3 may include one filar (e.g., to account for one missing conductor).

FIG. 8 illustrates an example embodiment of the lead 20 with a filler element 445 at the proximal end of the lead 20. In some embodiments, a proximal filler element may be included at the proximal portion to occupy at least a portion of the gaps at a proximal portion of the neurostimulation lead. For example, referencing FIG. 8, the filler element 445 is configured to occupy at least a portion of the gaps created as the conductors 520, 521, 522, and 523 exit the interior of the lead body at the proximal portion of the lead 20 (via the apertures 830, 831, 832, and 833, respectively) to couple with the connector interfaces 430, 431, 432, and 433, respectively. As discussed above with respect to the distal portion of the neurostimulation lead, gap sizes may vary across different segments of the proximal portion of the neurostimulation lead as conductors exit or terminate at different aperture points. For example, referencing FIG. 8, the segment P3 has the smallest gap size (resulting from one missing conductor 520), the segment P2 two has a larger gap size (resulting from two missing conductors 520 and 521), the segment Pl has an even larger gap size (resulting from three missing conductors 520, 521, and 522), and the segment PO has the largest gap size (resulting from for missing conductors 520, 521, 522, and 523).

As discussed above, a filler element may include only a single filar or alternatively may be a multi-filar structure. The filler elements (or filars making up the filler elements) may be of any suitable shape. For example, a filler element may include a flattened ribbon-like filar, which may be rectangular, tapered, triangular, elliptical, or any other suitable shape. As another example, the filler element may include a non-flattened filar, which may have a cross-section that is circular, rectangular, triangular, elliptical, or any other suitable shape. Filler elements (or filars making up the filler elements) may be of any suitable size or volume. In some embodiments, a filler element may include a filar having a length of 10 to 60 mm. In some embodiments, a filler element may include a filar having a diameter of 0.03 to 0.3 mm. In some embodiments, a filler element may include a ribbon-like filar (e.g., a rectangular ribbon similar to the filler element 440 illustrated in FIG. 7) having a width of 0.01 to 2 mm. The dimensions of filler elements may be selected based on the dimension of the conductors, the gap size, number of electrodes, electrode dimensions, electrode pitch, and/or length of the lead.

Filler elements may be composed of one or more electrically nonconductive materials, such that the filler elements are electrically nonconductive. For example, filler elements may be composed at least in part of a polymer material, such as a polyurethane material, a silicone material, high-density polyethylene (HDPE), poly carbonates, or a suitable plastic material. In some embodiments, filler elements may be short segments of inactive conductors (with insulative coatings). Using segments of conductors can help simply manufacturing and supply costs.

In some embodiments, filler elements used in the neurostimulation lead may be composed of substantially the same material as the surrounding lead body. For example, the lead body and a filler element disposed within the lead body may both be composed at least in part of a polymer material, such as a polyurethane material. Having the filler elements be the same material as the surrounding lead body may help with sealing apertures of the lead body during, for example, the reflow process described below.

In some embodiments, a neurostimulation lead may have a filler element only at the distal end of the neurostimulation lead. In other embodiments, a neurostimulation lead may have a filler element only at the proximal end of the neurostimulation lead. In other embodiments, a neurostimulation lead may have a filler element at both the proximal end and the distal end of the neurostimulation lead. In these embodiments, the filler element at the proximal end may be substantially of the same type as the filler element at the distal end, or alternatively may be different. For example, the filler element at one end may be a single filar structure and the filler element at the other end may be a multi-filar structure. As another example, the filler element at one end may be a coiled structure and the filler element at the other end may be an uncoiled structure (e.g., to conform to the configuration of the conductors when the conductors are coiled at one end and uncoiled at the other end).

In some embodiments, the neurostimulation lead may include any number of markers for aiding in locating the lead within a patient, for example during a lead positioning process or during a follow-up monitoring/check-up to ensure proper lead placement. In some embodiments, the neurostimulation lead may include one or more visual markers (e.g., radiopaque markers) that may be visible using an imaging system. For example, referencing FIG. 4, the lead 20 may include a number of markers such as the visual markers 470.

Experimental data supports the effectiveness of the use of filler elements in preventing or reducing current leakage. Three different types of leads were tested, and a summary of the testing data is shown in the tables below. The three different types of leads tested were: a conventional reference lead with no filler element (Table I), a lead according to the disclosure herein having a coiled rectangular ribbon-shaped polyurethane filler element (Table 2), and a lead according to the disclosure herein having a coiled bi-filar polyurethane filler element (Table 3). Testing was conducted by submerging the leads in a saline solution that mirrored electrolyte concentrations of body fluid that neurostimulation leads would typically be surrounded by when implanted within a patient. Once the leads were submerged, voltage measurements were taken across different pairs of electrode of the lead to measure resistances between the different pairs of electrodes. Each of the leads had four electrodes (E0, E1, E2, and E3) arranged linearly along the lead body. A resistance between the pairs of electrodes that is equal to or greater than 50 kQ may be an indication that the saline solution has not ingressed within the lead body to cause appreciable current leakage—that is, there is not an appreciable electrical pathway between the electrodes that would decrease the resistance between the electrodes (which is afforded by nonconductive polyurethane in the tested leads). By contrast, a resistance that is less than 50 kQ may be an indication that there is some current leakage, and the lower the resistance, the greater the current leakage. Referencing Table 1, there is a significant drop in resistance in the reference lead. For example, the resistance between E0 and E1 is 9.53 kQ, the resistance between E0 and E2 is 20.55 kQ, the resistance between E0 and E3 is 24.22 kQ, and the resistance between E1 and E2 is I 1.08 kQ. Referencing Tables 2 and 3 (which show data for leads including coiled polyurethane filler elements), the resistances between all of the tested pairs of electrodes is greater than 50 kQ, indicating that there is no appreciable current leakage due to ingress of saline solution. In this test, a passing grade was awarded for each pair of electrodes if it had a resistance greater than or equal to 25 kQ. As reflected in Table 1 below, none of the pairs of electrodes passed in the reference lead with no filler element, in direct contrast to the leads with filler elements (see Tables 2 and 3).

TABLE 1
Reference Lead with No Filler Element

| From | To | R | Pass? |
|---|---|---|---|
| (electrode) | (electrode) | [k!!] | (R:: 25 k!!) |
| 0 | 1 | 9.53 | NO |
| 0 | 2 | 20.55 | NO |
| 0 | 3 | 24.22 | NO |
| 1 | 2 | 11.08 | NO |
| 1 | 3 | 14.87 | NO |
| 2 | 3 | 8.37 | NO |

TABLE 2
Lead with Rectangular Ribbon Coil

| From | To | R | Pass? |
|---|---|---|---|
| (electrode) | (electrode) | [k!!] | (R:: 25 k!!) |
| 0 | 1 | 1000.00 | YES |
| 0 | 2 | 1000.00 | YES |
| 0 | 3 | 1000.00 | YES |
| 1 | 2 | 1000.00 | YES |
| 1 | 3 | 1000.00 | YES |
| 2 | 3 | 1000.00 | YES |

TABLE 3
Lead with Bifilar Coil

| From | To | R | Pass? |
|---|---|---|---|
| (electrode) | (electrode) | [k!!] | (R:: 25 k!!) |
| 0 | 1 | 1000.00 | YES |
| 0 | 2 | 1000.00 | YES |
| 0 | 3 | 1000.00 | YES |
| 1 | 2 | 1000.00 | YES |
| 1 | 3 | 1000.00 | YES |
| 2 | 3 | 1000.00 | YES |

IPGs and EPGs apply electrical energy for a neurostimulation from a power source (e.g., a battery) having a finite power supply. In many cases (e.g., in sacral neuromodulation use cases), IPGs once implanted are typically expected to have a lifespan of 5 to 15 years. As mentioned previously, current leakage over time causes the need to recalibrate periodically to adjust power output upward to account for the current leakage to maintain effectiveness of therapy. This takes a toll on battery life, and is particularly problematic for IPGs with nonrechargeable batteries, which would need to be surgically explanted for battery replacement. It is also problematic for IPGs that have rechargeable batteries, because it would require more frequent charging of the battery. This is not only an inconvenience for the patient, but the increased number of charging cycles may reduce the lifespan of the battery. Similarly, current leakage also affects external pulse generators EPGs, which would need to have batteries replaced or recharged more frequently than would be optimal.

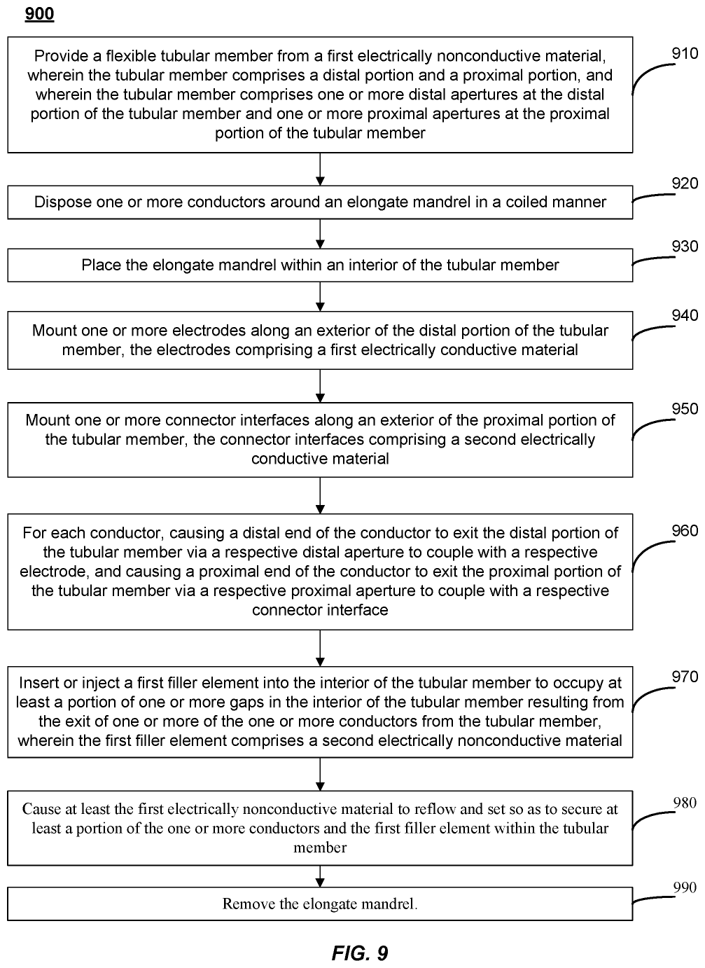

FIG. 9 illustrates an example method 900 of manufacturing a neurostimulation lead. Any suitable method may be used in manufacturing the neurostimulation leads described herein. Referencing FIG. 9, in some embodiments, the method of manufacture may include, at step 910, providing a flexible tubular member from a first electrically nonconductive material, wherein the tubular member comprises a distal portion and a proximal portion. This tubular member may be a hollow structure that forms the scaffolding for what will become the lead body when manufacturing is completed. The tubular member may include one or more distal apertures at the distal portion of the tubular member and may further include one or more proximal apertures at the proximal portion of the tubular member. In some embodiments, the manufacturing method may include forming the apertures via any suitable means. For example, the apertures may be formed by using a laser cutting method, by using a sharpened element, by molding the tubular member to include the apertures, etc. Referencing FIG. 6 for example, the apertures may be circular holes (e.g., the apertures 610, 611, 612, 613), rectangular openings, slits, etc. The tubular member may be composed of any suitable material, and may be electrically nonconductive. For example, the tubular member may be composed of a polymer material, such as a polyurethane material.

At step 920, the method may include disposing one or more conductors around an elongate mandrel in a coiled manner. A mandrel may be any elongate structure suitable for acting as a support for ultimately positioning conductors and filler elements within the tubular member. The conductors may be elongate, drawn-out wires. The conductors may be made of any conductive material (e.g., a metal that is suitably ductile and malleable to allow for drawing out the wires and adequate shaping). Each of the conductors may be surrounded by an insulative material or coating. In some embodiments, the conductors may be pre-coiled and slid onto the elongate mandrel. In other embodiments, the conductors may be substantially straight conductors that are coiled by winding the conductors around the elongate mandrel. In yet other embodiments, the method may not require coiled conductors, in which case the conductors may be maintained in an uncoiled, substantially straight manner.

At step 930, the method may include placing the elongate mandrel within an interior of the tubular member. At step 940, the method may include mounting one or more electrodes along an exterior of the distal portion of the tubular member, the electrodes comprising a first electrically conductive material. At step 950, the method may include mounting one or more connector interfaces along an exterior of the proximal portion of the tubular member, the connector interfaces comprising a second electrically conductive material. The electrodes and connector interfaces may be mounted by any suitable means. For example, they may be mounted using an adhesive, a bolt or screw mechanism, or simply a friction fit.

At step 960, the method may include for each conductor, causing a distal end of the conductor to exit the distal portion of the tubular member via a respective distal aperture to couple with a respective electrode. This step may also include causing a proximal end of the conductor to exit the proximal portion of the tubular member via a respective proximal aperture to couple with a respective connector interface. In some embodiments, the distal ends of the conductors may be coupled directly to one or more respective electrodes (e.g., by way of welding) or coupled indirectly to one or more respective electrodes (e.g., via one or more components). For example, referencing FIG. 7, the conductor 523 may be welded to the electrode 413, the conductor 522 may be welded to the electrode 412, the conductor 521 may be welded to the electrode 411, and the conductor 510 may be welded to the electrode 410. The conductors may be coupled to the connector interfaces in a similar manner.

At step 970, the method may include inserting a first filler element into the interior of the tubular member to occupy at least a portion of one or more gaps in the interior of the tubular member resulting from the exit of one or more of the one or more conductors from the tubular member, wherein the first filler element comprises a second electrically nonconductive material. In some embodiments, the first filler element may be inserted at the distal portion of the tubular member, and a second filler element may be inserted at the proximal portion of the tubular member. In some embodiments, the filler elements may be caused to be coiled, particularly when the conductors are coiled. In these embodiments, the conductors may be coiled at a first pitch, and the filler elements may be coiled at a second pitch. The first pitch may substantially be the same as the second pitch, or may alternatively be different from the second pitch. In embodiments where the filler elements are coiled, the filler elements may be pre-coiled and slid onto the elongate mandrel. In other embodiments, the conductors may be substantially straight conductors that are coiled by winding the conductors around the elongate mandrel. The filler elements may be inserted into the tubular member after the elongate mandrel has already been inserted (and then slid or coiled around the elongate mandrel). Alternatively, the filler elements may be slid or coiled around the elongate mandrel prior to insertion of the elongate mandrel. For example, the conductors and filler elements may be positioned on or around the elongate mandrel prior to placing the elongate mandrel within the interior of the tubular member.

In the case of a filler element that is a multi-filar structure, the fillers of the multi-filar structure may be compiled together (e.g., bonded to each other) prior to placing the multi-filar structure within the lead body. Alternatively, each of the filars may be inserted into the lead body separately. For example, referencing FIG. 7, a first filar may be inserted to extend from the distal tip 450 to the aperture 613; a second filar having a smaller length may be inserted to extend from the distal tip 450 to the aperture 612; a third filar having an even smaller length may be inserted to extend from the distal tip 450 the aperture 611; and a fourth filar having an even smaller length may be inserted to extend from the distal tip 450 to the aperture 610. The resulting filler element has varying thickness among the different segments DO, DI, D2, and D3.

In some embodiments, when the lead includes two or more electrodes, the method may include placing one or more spacers in between the electrodes and connector interfaces. The spacers may be any suitable structural element that is capable of being placed (e.g., on or around the exterior of the lead body) so as to appropriately space out the electrodes at desired locations along the lead body. The spacers may be made of any suitable material that makes the spacers electrically nonconductive. For example, the spacers may be made of a polymer material such as a polyurethane material. In some embodiments, the spacers may be made of substantially the same material as the tubular member (e.g., a polymer material such as polyurethane). In some embodiments, the spacers, the filler element, and the tubular member may all be made of substantially the same material (e.g., a polymer material such as polyurethane). The spacers may be placed prior to mounting the electrodes and connector interfaces or may alternatively be placed after mounting the electrodes and connector interfaces.

At step 980, the method may include causing at least the first electrically nonconductive material to reflow and set so as to secure at least a portion of the one or more conductors and the first filler element within the tubular member. The reflow process may involve heating the assembled components of the lead to a suitable temperature so as to melt materials of particular elements of the lead, such as the tubular member and/or the filler element (and not other elements such as the conductors, the electrodes, the connector interfaces, etc.). When the melted materials cool down and set, a seal is formed that may secure various elements of the lead, such as the conductors, the filler elements, etc., within the lead body. This reflow step may also cause the melted material to flow around the conductors at the exit points to fill in any gaps in the apertures. In some embodiments, when spacers are used as described above, the reflow step also causes the material of the spacer to reflow and set so as to further aid with sealing the apertures along the lead body.

At step 990, the method may include removing the elongate mandrel, which may leave behind a lumen, which may be configured to receive a stylet to aid with the implantation procedure, as described above.

Particular embodiments may repeat one or more steps of the method of FIG. 9, where appropriate. Although this disclosure describes and illustrates particular steps of the method of FIG. 9 as occurring in a particular order, this disclosure contemplates any suitable steps of the method of FIG. 9 occurring in any suitable order. For example, the electrodes and connector interfaces may be mounted to the tubular member prior to placing the elongate mandrel within the tubular member. As another example, the filler element may be inserted prior to placing the elongate mandrel within the tubular member. Moreover, although this disclosure describes and illustrates an example method for manufacturing a neurostimulation lead, including the particular steps of the method of FIG. 9, this disclosure contemplates any suitable method for manufacturing a neurostimulation lead, including any suitable steps, which may include all, some, or none of the steps of the method of FIG. 9, where appropriate. Furthermore, although this disclosure describes and illustrates particular components, devices, or systems carrying out particular steps of the method of FIG. 9, this disclosure contemplates any suitable combination of any suitable components, devices, or systems carrying out any suitable steps of the method of FIG. 9.

What is claimed is:

1. A method of manufacturing an implantable neurostimulation lead, the method comprising:
providing a flexible tubular member from a first electrically nonconductive material, wherein the tubular member comprises a distal portion and a proximal portion, and wherein the tubular member comprises one or more distal apertures at the distal portion of the tubular member and one or more proximal apertures at the proximal portion of the tubular member; disposing one or more conductors around an elongate mandrel in a coiled manner;
placing the elongate mandrel within an interior of the tubular member;
mounting one or more electrodes along an exterior of the distal portion of the tubular member, the electrodes comprising a first electrically conductive material;
mounting one or more connector interfaces along an exterior of the proximal portion of the tubular member, the connector interfaces comprising a second electrically conductive material;
for each conductor, causing a distal end of the conductor to exit the distal portion of the tubular member via a respective distal aperture to couple with a respective electrode, and causing a proximal end of the conductor to exit the proximal portion of the tubular member via a respective proximal aperture to couple with a respective connector interface;
inserting or injecting a first filler element into the interior of the tubular member to occupy at least a portion of one or more gaps in the interior of the tubular member resulting from the exit of one or more of the one or more conductors from the tubular member, wherein the first filler element comprises a second electrically nonconductive material; and
removing the elongate mandrel.

2. The method of claim 1, wherein disposing one or more conductors around the elongate mandrel in a coiled manner comprises coiling the one or more conductors around the elongate mandrel at a first pitch.

3. The method of claim 2, wherein inserting or injecting the first filler element into the interior of the tubular member comprises coiling the first filler element around the elongate mandrel at a second pitch, the second pitch substantially the same as the first pitch.

4. The method of claim 2, wherein inserting or injecting the first filler element into the interior of the tubular member comprises coiling the first filler element around the elongate mandrel at a second pitch, the second pitch different from the first pitch.

5. The method of claim 1, wherein the one or more conductors are pre-coiled, and wherein disposing one or more conductors around the elongate mandrel in a coiled manner comprises sliding the pre-coiled conductors over the elongate mandrel.

6. The method of claim 5, wherein the first filler element is pre-coiled, and wherein inserting or injecting the first filler element into the interior of the tubular member comprises sliding the pre-coiled filler element over the elongate mandrel.

7. The method of claim 1, further comprising causing at least the first electrically nonconductive material to reflow and set so as to secure at least a portion of the one or more conductors and the first filler element within the tubular member.

8. The method of claim 7, wherein the one or more electrodes comprises a first electrode and a second electrode, and wherein the one or more connector interfaces comprises a first connector interface and a second connector interface, the method further comprising:
placing a first spacer in between the first electrode and the second electrode;
placing a second spacer in between the first connector interface and the second connector interface, wherein the first spacer and the second spacer comprise a third electrically nonconductive material; and
causing the third electrically nonconductive material to reflow and set so as to seal the one or more distal apertures and the one or more proximal apertures around the distal and proximal ends of the one or more conductors.

9. The method of claim 8, wherein the first electrically nonconductive material and the third electrically nonconductive material are substantially the same.

10. The method of claim 9, wherein the first electrically nonconductive material, the second electrically nonconductive material, and the third electrically nonconductive material comprise a polyurethane material.

11. The method of claim 1, wherein the first filler element is a multi-filar structure, and wherein inserting or injecting the first filler element into the interior of the tubular member comprises:
inserting a first filar into the interior of the tubular member; and
inserting a second filar into the interior of the tubular member.

12. The method of claim 1, further comprising, for each conductor:
welding the distal end of the conductor to one or more respective electrodes; and
welding the proximal end of the conductor to one or more respective connector interfaces.

13. The method of claim 1, further comprising forming the one or more distal apertures and the one or more proximal apertures by creating slits in the tubular member.

14. The method of claim 1, wherein the first filler element is inserted or injected at the distal portion of the tubular member, and wherein the method further comprises inserting or injecting a second filler element at the proximal portion of the tubular member, the second filler element comprising a fourth electrically nonconductive material.

15. The method of claim 1, wherein the first filler element comprises a solid extrusion, wherein inserting or injecting comprises inserting the solid extrusion into the interior of the tubular body and the method further comprises securing at least a portion of the one or more conductors and the solid extrusion within the tubular member.

16. The method of claim 1, wherein the first filler element comprises an adhesive or cured thermoplastic or thermoset polymer material, wherein inserting or injecting comprises injecting the polymer material into the interior of the tubular body so as to form a seal to secure at least a portion of the one or more conductors and the first filler element within the tubular member.

17. The method of claim 1, further comprising removing excess material in the interior of the tubular member, on the exterior of the tubular member, or an exterior of the one or more electrodes by machining, grinding, or polishing.

18. A method of manufacturing an implantable neurostimulation lead, the method comprising:
incorporating a filler element into an interior of a flexible tubular member to occupy at least a portion of one or more gaps in the interior of the tubular member resulting from an exit of one or more conductors from the tubular member via a respective distal aperture to couple with a respective electrode,
wherein the flexible tubular member comprises a first electrically nonconductive material and the filler element comprises a second electrically nonconductive material.

19. The method of claim 18, wherein the incorporating step comprises inserting, injecting, molding, machining, 2D printing, 3D printing, melt extruding, solid-state forming, casting, vacuum forming, or coating the filler element into the interior of the flexible tubular member.

20. The method of claim 18, wherein the first electrically nonconductive material and the second electrically nonconductive material comprise a polyurethane material.
